SEQUENCE LISTING (4 sequences)

SEQ ID NO: 1 - DNA, 34 bp, Artificial Sequence
  Description of Artificial Sequence Synthetic primer
agcttacgacaaaatgcacaaattcacaaaattt

SEQ ID NO: 2 - DNA, 35 bp, Artificial Sequence
  Description of Artificial Sequence Synthetic primer
gcgtcattattctgatggttatctttttgagaggt

SEQ ID NO: 3 - DNA, 28 bp, Artificial Sequence
  Description of Artificial Sequence Synthetic primer
actttcccacacttgataaatatcctcg

SEQ ID NO: 4 - DNA, 28 bp, Artificial Sequence
  Description of Artificial Sequence Synthetic primer
ataatcgttttcaaccgcaaaattacag

(19) United States
(12) Patent Application Publication
(10) Pub. No.: US 2008/0070283 A1
(43) Pub. Date: Mar. 20, 2008

(54) DNA FRAGMENT AMPLIFICATION METHOD, REACTION APPARATUS FOR AMPLIFYING DNA FRAGMENT AND PROCESS FOR PRODUCING SAME

(75) Inventors: Toru Sano, Tokyo (JP); Masakazu Baba, Tokyo (JP); Kazuhiro Ilda, Tokyo (JP); Hisao Kawaura, Tokyo (JP); Noriyuki Iguchi, Tokyo (JP); Wataru Hattori, Tokyo (JP); Hiroko Someya, Tokyo (JP)

Correspondence Address: FOLEY AND LARDNER LLP;SUITE 500, 3000 K STREET NW, WASHINGTON, DC, 20007, US

(73) Assignee: NEC CORPORATION

(21) Appl. No.: 11/854,315

(22) Filed: Sep. 12, 2007

Related U.S. Application Data
(63) Continuation of application No. 10/548,590, filed on Sep. 12, 2005, now abandoned.

(30) Foreign Application Priority Data
Mar. 18, 2003 (JP) 2003-073095

(51) Int. Cl. C12P 19/34; C12M 1/00

(52) U.S. Cl. 435/91.2; 435/289.1

(57) ABSTRACT

A reaction apparatus (10) includes a substrate (12) and a plurality of columnar members (14) formed on the substrate (12). Oligonucleotides for immobilization (16) having sequences complementary to sequences of both ends of a starting template DNA (18) is adhered on the surfaces of the substrate (12) and the columnar members (14). The starting template DNA (18) can be immobilized over the adjacent columnar members (14) by introducing the starting template DNA (18) under the elongated condition. PCR is conducted in such condition.

### CROSS-REFERENCE TO RELATED PATENT APPLICATIONS

Japan Priority Application 2003-073095, filed Mar. 18, 2003 including the specification, drawings, claims and abstract, is incorporated herein by reference in its entirety. This application is a Continuation of U.S. application Ser. No. 10/548,590 (National Stage of PCT/JP2004/003575), filed Sep. 12, 2005, incorporated herein by reference in its entirety.

### TECHNICAL FIELD

The present invention relates to a reaction apparatus for amplifying a relatively long DNA fragment and a process for producing thereof.

### BACKGROUND ART

Polymerase chain reaction (PCR) is known as a process for amplifying specified DNA fragment (see, for example, U.S. Pat. No. 4,683,195). In an ordinary PCR process, a target DNA is, first, thermally denatured to form a single strand DNA, and a primer having a base sequence complementary to a base sequence of an end of the DNA is bound to the obtained single strand DNA via an annealing. Thereafter, a elongation reaction for a complementary strand DNA is conducted by employing a DNA polymerase, and such cycle is repeated to exponentially amplify the target DNA.

Conventionally, for the purpose of conducting an observation of chain polymer molecule such as DNA, employing a scanning tunneling microscope under an elongated condition without applying an electric field thereto, a technology is disclosed, in which a solution is heated while one end of DNA is in contact with an electrode and the other end thereof is elongated perpendicularly to the electrode, thereby bonding and fixing the molecule to a substrate while being elongated (see, for example, Japan Patent No. 3,064,001).

### DISCLOSURE OF THE INVENTION

However, it is difficult in the conventional PCR to amplify relatively longer DNA fragment of, for example, several tens kb or higher as a template via PCR.

In view of the above-described circumstances, it is an object of the present invention to provide a technology to provide an effective amplification even in the case of employing a template of longer DNA fragment.

Inventors of the present invention have considered that a contribution to the fact that a PCR process for a template of relatively longer DNA fragment of, for example, several tens kb or higher cannot be effectively conducted is that excessively longer DNA fragment of the template tends to be twisted, and for that reason, a elongation reaction for a complementary strand DNA is interrupted, thereby leading to the development of the present invention.

Since a long DNA fragment employed for a starting template such as, for example, chromosomal DNA, or a DNA fragment broken by ultrasonic wave contains a number of sequences other than the target fraction for amplification, a larger torsion is occurred therein, and thus it will be a barrier for amplifying the DNA. It is considered that, in such case, a elongation reaction of a primary complementary strand can be conducted with higher efficiency by preventing such torsion of the DNA fragment.

According to the present invention, there is provided a method for amplifying a DNA fragment, comprising: binding the DNA fragment to a binding site formed on a surface of a base member; and synthesizing a complementary strand of the DNA fragment by using the DNA fragment as template under a status of the DNA fragment being bound to the surface of the base member.

Since this provides the condition, in which the DNA fragment used as the template is bound and immobilized to the surface of the base member when the complementary strand of the DNA fragment is synthesized, the torsion of the DNA fragment can be reduced even if a relatively longer DNA fragment is employed as the template, thereby preferably conducting the synthesis of the complementary strand.

The method for amplifying the DNA fragment according to the present invention may have a configuration, in which the binding site is configured to be bound to a DNA sequence, which is located in the outside of an amplifying target region of an amplifying target DNA fragment.

Here, the “outside” indicates portions of the amplifying target DNA fragment except the amplifying target regions. Both of the outsides of the amplifying target region amplifying target DNA fragment may be bound to the surface of the base member, or only one outside thereof may also be bound to the surface of the base member. When only one outside thereof is bound to the surface of the base member, it is preferable to conduct the synthesis of the complementary strand under a status of elongating the DNA fragment by, for example, creating some flow rate in the reaction field during the synthesis of the complementary strand. Having such configuration, the torsion of the DNA fragment can be reduced, thereby providing better synthesis of the complementary strand.

The method for amplifying the DNA fragment according to the present invention may have a configuration, in which the binding site may include an oligonucleotide for immobilization having a sequence complementary to a portion of an amplifying target DNA fragment.

Having such configuration, a portion of the amplifying target DNA fragment is bound to the oligonucleotide for immobilization at the binding site via hydrogen bond, so that the DNA fragment can be bound to the binding site.

The method for amplifying the DNA fragment according to the present invention may have a configuration, in which the binding site contains two or more types of oligonucleotides for immobilization, the oligonucleotides having sequences complementary to DNA sequences located in the both outsides of an amplifying target region of an amplifying target DNA fragment.

Having such configuration, both outsides of the amplifying target region of an amplifying target DNA fragment are bound to oligonucleotides for immobilization in the binding sites via hydrogen bond, thereby allowing the DNA fragment being bound to the binding sites via two points.

The method for amplifying the DNA fragment according to the present invention may have a configuration, in which the DNA fragment is bound to the surface of the base member under an elongated condition in the process for binding thereof.

Having such configuration, the torsion of the DNA fragment can be reduced, so that the synthesis of the complementary strand can be suitably conducted.

The method for amplifying the DNA fragment according to the present invention may have a configuration, in which the DNA fragment is bound to the surface of the base member under an elongated condition by utilizing, for example, shearing stress in the process for binding thereof. A method of producing a flow rate in a reaction field produce, or a method of providing a rotation to the reaction field may be utilized to provide shearing stress.

The method for amplifying the DNA fragment according to the present invention may have a configuration, in which the DNA fragment is bound to the surface of the base member under an elongated condition by applying a low frequency electric field over the DNA fragment in the process for binding thereof. Here, the low frequency electric field may be, for example, an electric field of equal to or lower than 100 Hz.

The method for amplifying the DNA fragment according to the present invention may have a configuration, in which the DNA fragment is bound to the surface of the base member under a condition of being elongated by applying a high electric field over the DNA fragment in the process for binding thereof. Here, the high electric field may be, for example, an electric field of equal to or higher than 500 kHz.

The method for amplifying the DNA fragment according to the present invention may have a configuration, which further comprises stretching the surface of the base member after the binding the DNA fragment.

Having such configuration, the torsion of the DNA fragment can be reduced, so that the synthesis of the complementary strand can be suitably conducted.

The method for amplifying the DNA fragment according to the present invention may have a configuration, in which the synthesizing the complementary strand includes denaturing and separating the template DNA fragment and the complementary strand, wherein in the binding the DNA fragment, the DNA fragment is immobilized to the binding site so that the DNA fragment is not separated from the binding site in the denaturing and separating.

In the denaturing and separating the template DNA fragment and the complementary strand, a heat of about 95 degree C. is normally added. Even if such heat of the temperature is added in the method for amplifying the DNA fragment according to the present invention, it is preferred to immobilize the DNA fragment to the binding site so as to avoid separating the DNA fragment from the binding site. For example, when the binding site includes the oligonucleotide for immobilization stated above, providing a bond of the DNA fragment with the binding site via hydrogen bond will not be possibly enough to avoid deactivating the bond of the DNA fragment with the binding site in the denaturing and separating the template DNA fragment and the complementary strand. In order to avoid deactivating the bond of the DNA fragment with the binding site in such case in the present invention, the DNA fragment and binding site may be immobilized via, for example, covalent bond.

The method for amplifying the DNA fragment according to the present invention may have a configuration, in which the amplifying target DNA fragment has a length of equal to or larger than 10 kb.

While the torsion of the DNA fragment may be possibly occurred in the conventional amplifying method when such a relatively longer DNA fragment is employed as a template, according to the method for amplifying the DNA fragment of the present invention, the torsion of DNA fragment can be inhibited to provide better amplifying reaction even though a relatively longer DNA fragment is employed as a template.

The method for amplifying the DNA fragment according to the present invention may have a configuration, in which the binding site is configured to be bound to a portion of the amplifying target region of the amplifying target DNA fragment, and the method further including, in the synthesizing the complementary strand, deactivating the bond of the DNA fragment with the binding site. As the method for deactivating the bond of the DNA fragment with the binding site, a heat of around 75 degree C. to 85 degree C. may be added. Although a heat of on the order of about 90 degree C. is added when the template DNA fragment and the complementary strand are denatured and separated, as have been described above, number of the bases of the DNA binding thereto is very small in the bond of the binding site with the DNA fragment, as compared with the bond of the DNA fragment with the complementary strand. Smaller number of bases in the DNA bound thereto provides weaker binding strength between the double strand as compared with the case of having larger number of the base in the DNA, and thus, when a heat of around 75 degree C. to 85 degree C. is added, the bond of the binding site with the DNA fragment can be deactivated, though the template DNA is not separated from the complementary strand in an elongating state. A sequence complementary to the bound portion thereof may also be synthesized by separating the template DNA fragment from the binding site, in a stage that the complementary strand of the template DNA fragment is elongated to a certain extent. Since this provides the bound portion included in the synthesized complementary strand, the complementary strand can be immobilized to the surface of the base member, thereby achieving the amplification of the relatively longer DNA fragment with an improved efficiency.

According another aspect of the present invention, there is provided a reaction apparatus for conducting an amplifying of a DNA fragment, comprising: a surface of a base member; and binding sites that are formed on the surface of the base member and are capable of being bound to amplifying target DNA fragments.

Since the DNA fragment is bound and immobilized to the binding site by introducing an amplifying target DNA fragment into thus configured reaction apparatus, the torsion of the DNA fragment is inhibited to provide better amplification of the DNA fragment even though a relatively longer DNA fragment is employed as the template.

The reaction apparatus according to the present invention may have a configuration, in which the binding sites are formed on a plurality of regions provided with certain distances therebetween. Here, the distance between respective regions may preferably be on the order of the same length as the length of the amplifying target portion of the amplifying target DNA fragment. Having such configuration, the DNA fragment can be immobilized to the surface of the base member under the status of elongating the DNA fragment to a certain extent, by providing a biding of the DNA sequence located in the outside of the amplifying target portion of the DNA fragment to the binding site, and therefore the torsion of the DNA fragment can be reduced.

The reaction apparatus according to the present invention may have a configuration, in which the binding site includes a plurality of protruding portions formed on the surface of the base member. Here, the protruding portion may be a columnar member formed by a fine processing. The distances between the protruding portions may preferably be on the order of the same length as the length of the amplifying target portion of the amplifying target DNA fragment. Having such configuration, the DNA fragment can be immobilized to the surface of the base member under the status of elongating the DNA fragment to a certain extent, by providing a biding of the DNA sequence located in the outside of the amplifying target portion of the DNA fragment to the binding site, and therefore the torsion of the DNA fragment can be reduced. Further, the DNA fragment can be caught on the protruding portion to promote the immobilization by providing the protruding portion to the binding site.

The reaction apparatus according to the present invention may have a configuration, in which the binding site is bound to both outsides of the amplifying target DNA fragment. Having such configuration, the DNA fragment can be immobilized to the surface of the base member under the status of elongating the DNA fragment to a certain extent, and therefore the torsion of the DNA fragment can be reduced.

The reaction apparatus according to the present invention may have a configuration, in which the binding site includes an oligonucleotide for immobilization having a sequence complementary to a portion of the amplifying target DNA fragment.

The reaction apparatus according to the present invention may have a configuration, in which the base member is composed of a material that is capable of being stretched. The base member may be composed of a rubber or a plastic material, for example.

Further, the reaction apparatus according to the present invention may further comprise a unit that can be utilized to immobilize the DNA fragment under an elongated condition. Typical examples of such unit may include an electric field-applying unit that applies low frequency electric field and high electric field and a shearing stress-applying unit that applies shearing stress to the reaction field. Exemplified shearing stress-applying units may include a stirring member, for example, which produces a flow rate in the reaction field, a spinning unit that provides a spinning of the reaction vessel, or the like.

According further aspect of the present invention, there is provided a method for manufacturing a reaction apparatus for conducting an amplification of a DNA fragment, comprising: forming a binding site, which is to be bound to an amplifying target DNA fragment, on a surface of a base member; and binding the amplifying target DNA fragment to the binding site.

The method for manufacturing a reaction apparatus according to the present invention may have a configuration, in which an oligonucleotide for immobilization having a sequence complementary to a portion of the amplifying target DNA fragment is immobilized on the surface of the base member in the forming the binding site.

The method for manufacturing a reaction apparatus according to the present invention may have a configuration, in which the DNA fragment is bound to the surface of the base member under an elongated condition in the process for binding the DNA fragment.

The method for manufacturing a reaction apparatus according to the present invention may further comprise stretching the surface of the base member after the binding the DNA fragment.

According to the present invention, better amplification of the DNA can be achieved even though a relatively longer DNA fragment is employed as the template.

### BRIEF DESCRIPTION OF THE DRAWINGS

The above and other objects, advantages and features of the present invention will be more apparent from the following description of the preferred embodiments taken in conjunction with the accompanying drawings.

FIG. 1 is flow chart, showing a procedure of PCR in the embodiment of the present invention.

FIG. 2 includes perspective views, illustrating a process for manufacturing a reaction apparatus in the embodiment of the present invention.

FIG. 3 includes diagrams, illustrating an example of a starting template DNA employed in the embodiment of the present invention.

FIG. 4 includes diagrams, illustrating a method for forming a columnar member of a reaction apparatus in the embodiment of the present invention.

FIG. 5 includes diagrams, illustrating a method for forming a columnar member of a reaction apparatus in the embodiment of the present invention.

FIG. 6 includes diagrams, illustrating a method for forming a columnar member of a reaction apparatus in the embodiment of the present invention.

FIG. 7 includes diagrams, illustrating a process for manufacturing a reaction apparatus in the embodiment of the present invention.

FIG. 8 includes diagrams, illustrating a configuration of the reaction apparatus in the embodiment of the present invention.

FIG. 9 includes diagrams, illustrating a process for manufacturing the reaction apparatus in the embodiment of the present invention.

FIG. 10 includes diagrams, illustrating a method for immobilizing a starting template DNA in the embodiment of the present invention.

FIG. 11 includes diagrams, illustrating the reaction apparatus in the embodiment of the present invention.

FIG. 12 includes diagrams, illustrating another reaction apparatus in the embodiment of the present invention.

FIG. 13 includes diagrams, illustrating a method for amplifying a starting template DNA in the embodiment of the present invention.

FIG. 14 is a conceptual diagram, illustrating a method for immobilizing a DNA strand in the embodiment of the present invention.

FIG. 15 includes process diagrams, illustrating a method for manufacturing a substrate in the embodiment of the present invention.

### BEST MODE FOR CARRYING OUT THE INVENTION

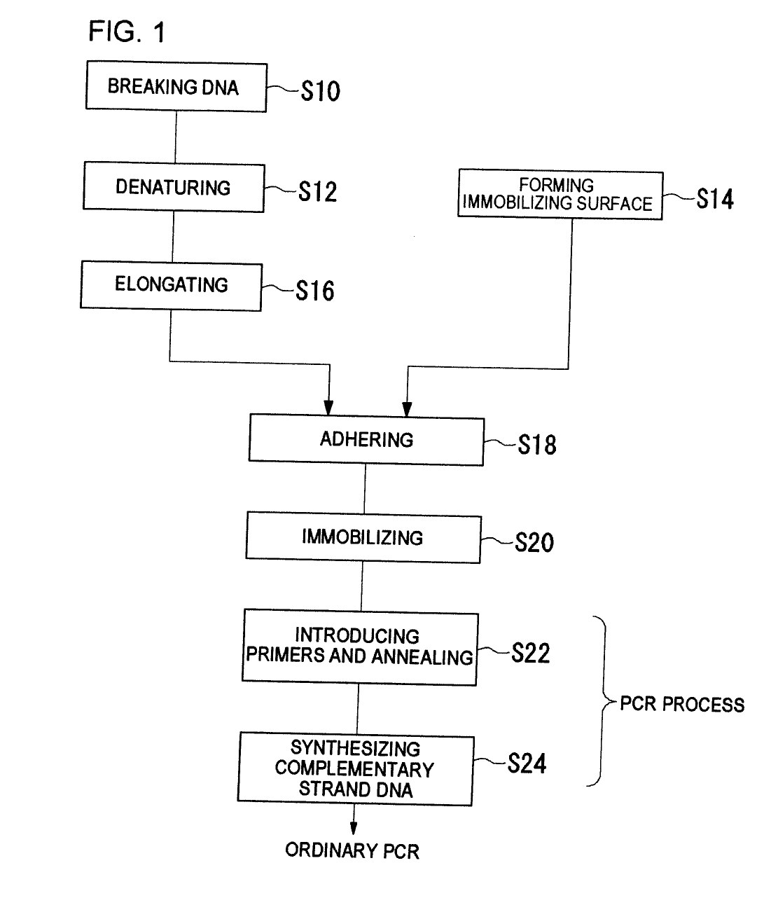

FIG. 1 is flow chart, showing a procedure of a PCR in the embodiment of the present invention.

First, a chromosomal DNA having an amplifying target portion is broken via ultrasonic wave to obtain a fragment containing a starting template DNA (S10). Although the chromosomal DNA is randomly broken in this occasion, the breaking is conducted so that a fragment includes an amplifying target portion and portions for immobilization located in the both outsides of the amplifying target portion. Such fragment functions as a starting template DNA. In the present embodiment, a primary complementary strand of the amplifying target portion of the starting template DNA is synthesized using the starting template DNA as the template, and thereafter, corresponding complementary strands are sequentially synthesized using the synthesized complementary strand as the template. Since the starting template DNA is obtained by being randomly broken, this includes base sequences other than the amplifying target portion, and thus has longer structure. Therefore, it is difficult to utilize the starting template DNA as the template as it is. In the present embodiment, after the starting template DNA is immobilized to synthesize the complementary strand, the obtained complementary strand is composed of only the amplifying target portion, and thus the corresponding complementary strand can be effectively synthesized without additional immobilization. Having this procedure, the amplifying target portion of the starting template DNA can be sub-exponentially amplified.

Next, the starting template DNA fragment is denatured by utilizing an alkali or a heat to modify the double stranded-starting template DNA fragment into the single strand product (S12). On the other hand, an immobilizing surface for immobilizing the starting template DNA fragment is formed (S14). An oligonucleotide for immobilization that is utilized to form a chemical bond with the starting template DNA fragment is immobilized on the immobilizing surface. The oligonucleotide for immobilization has a sequence complementary to the portion for immobilization in the starting template DNA. The method for forming the immobilizing surface will be described later in the respective embodiments.

Subsequently, the starting template DNA fragment that has been modified to the single strand is adhered to an oligonucleotide for immobilization of the immobilizing surface via chemical bond (S18).

Since the oligonucleotide for immobilization has a sequence complementary to the portion for immobilization in the starting template DNA fragment in this case, the starting template DNA fragment and the oligonucleotide for immobilization form hydrogen bond. Then, the starting template DNA fragment is immobilized to the immobilizing surface, so that the breakage of the bond of the starting template DNA fragment with the oligonucleotide for immobilization is avoided when heat is added in the subsequent PCR process (S20). Here, the starting template DNA fragment can be bound with the oligonucleotide for immobilization via covalent bond by employing a cross linker agent such as psoralen, for example.

In this occasion, the starting template DNA may be adhered to the oligonucleotide for immobilization under the elongated condition (S16), or the starting template DNA may be elongated after being immobilized to the oligonucleotide for immobilization, to obtain the condition that the starting template DNA is elongated, and the following PCR process is conducted under such condition.

First, an immobilizing surface is introduced into a reaction vessel for PCR. Then, a reaction solution is prepared by mixing specified quantities of a buffer solution for PCR, primers (sense primer, antisense primer), heat-resistant DNA polymerase and deoxyribonucleotide triphosphate (dNTP: mixture of dATP (2′-deoxyadenosine 5′-triphosphate), dGTP (2′-deoxyguanosine 5′-triphosphate), dCTP (2′-deoxycytidine 5′-triphosphate) and dTTP (2′-deoxythymidine 5′-triphosphate)) and is introduced in the reaction vessel. Subsequently, a heat of, for example, about 50 degree C. to 70 degree C., depending on a melting temperature of the oligonucleotide for the primer, is added to bind the starting template DNA fragment to the primer via an annealing (S22). Then, a reaction is conducted at, for example, about 70 degree C., depending upon the most suitable reaction temperature of the employed heat-resistant DNA polymerase, to synthesize a complementary strand DNA to the starting template DNA fragment by utilizing the heat-resistant DNA polymerase (S24).

Then, a heat of, for example, about 95 degree C. is added to denature the synthesized starting template DNA fragment and the complementary strand DNA to obtain a single strand, and then, the ordinary PCR process including the annealing, the elongation of the complementary strand and the denaturation is repeated. In addition, dual-temperature reaction system may also be utilized, depending on the property of the employed heat-resistant DNA polymerase.

Since the starting template DNA fragment is immobilized onto the immobilizing surface under the elongated condition in the embodiment of the present invention to conduct by PCR process, torsion of the DNA fragment is reduced to provide better amplification of the DNA even though the starting template DNA fragment is longer.

### FIRST EMBODIMENT

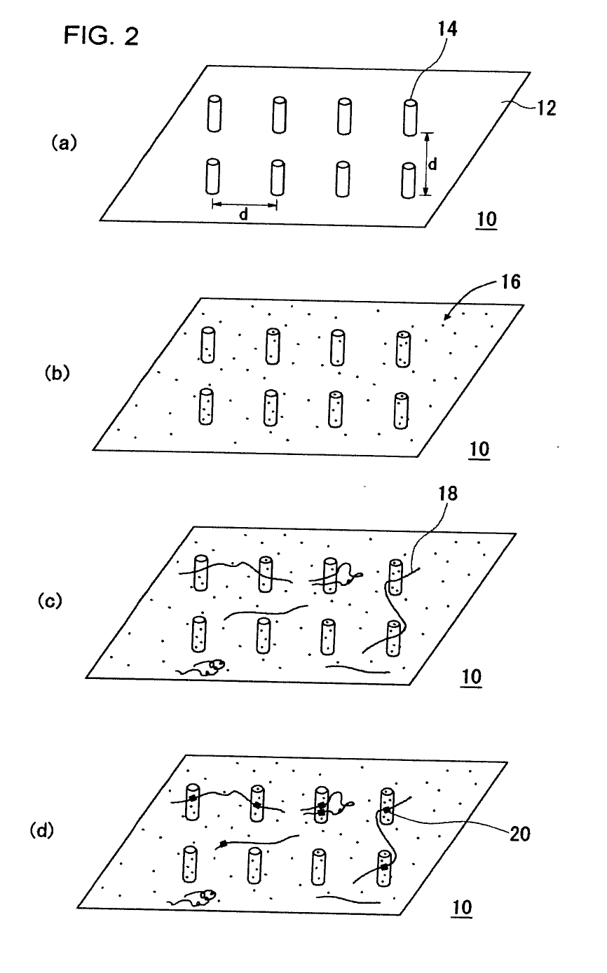

FIG. 2 is a perspective view, illustrating a process for manufacturing a reaction apparatus 10 in the present embodiment.

The reaction apparatus 10 comprises a substrate 12 and a plurality of columnar member 14 formed on the substrate 12 (FIG. 2(a)). While the substrate 12 is illustrated as a plane, the region having the columnar members 14 formed on the substrate 12 may be formed to have concave portions, so as to configure to be capable of introducing various types of reagents in the substrate 12. In addition, various types of reagents may also be introduced in the reaction vessel under the condition of introducing the substrate 12 into the reaction vessel.

While the columnar member 14 is shown as a cylinder, any types of geometries can be employed provided that a protruding portion, on which the starting template DNA fragment can be immobilized, is formed, such as: quasi-cylinder geometries such as cylindroid; cones such as circular cone, elliptical cone, trigonal pyramid and quadrangular pyramid; rectangular columns such as triangle column and square column; and additionally a stripe-shaped protrusion or the like. A distance d of respective columnar members 14 may be equivalent to a distance between portions for immobilization provided on both outsides of the amplifying target portion of the starting template DNA under the condition of elongating the starting template DNA, or slightly shorter than the distance between portions for immobilization. The length of the starting template DNA may be, for example, 2 to 100 kb. The length of DNA for 1 bp is about 0.33 nm, and therefore the distances d of respective columnar members 14 may be, for example, about 600 nm to 35 μm.

The substrate 12 may be composed of an elastic material, such as silicon, glass, quartz, various types of plastic materials, rubber and the like. As for the plastic materials, those having better processability are preferably employed, and exemplary plastic materials may include, for example, thermoplastic resins such as poly(methyl methacrylate) (PMMA), polyethylene terephthalate (PET), polycarbonate (PC) and the like, or thermosetting resins such as epoxy resin. The surfaces of the substrate 12 and the columnar members 14 may be coated with a metal such as gold (Au) or the like. It is preferable that the surfaces of the substrate 12 and the columnar members 14 are maintained to be clean. When the substrate 12 is composed of silicon, the surfaces of the substrate 12 and the columnar members 14 may be in a condition of being coated with silicon oxide films (SiO₂).

The oligonucleotide for immobilization 16 having the sequence complementary to the portion for immobilization in the starting template DNA is adhered to the thus configured surfaces of the substrate 12 and the columnar members 14 (FIG. 2(b)). When the substrate 12 is composed of glass, or when the surfaces of the substrate 12 and the columnar members 14 is coated with silicon oxide films, or coated with a metal, the oligonucleotide for immobilization 16 can be adsorbed to the surfaces of the substrate 12 and the columnar members 14 by maintaining the surfaces of the substrate 12 and the columnar members 14 being clean.

Descriptions will be made as follows, in reference to a case of using the substrate 12 composed of silicon. In this case, available oligonucleotide for immobilization 16 may be, for example, that contains thiol group to five prime end (5′ end). In this case, a chemical compound, which is capable of being bound to thiol, is immobilized onto the surfaces of the substrate 12 and the columnar members 14 in advance. The method for immobilizing such chemical compound will be described. First, the substrate 12 is immersed within, for example, a mixed solution of conc. HCl:CH₃OH at a mixing ratio of 1:1 for about 30 minutes, and then rinsed with distilled water, and thereafter, is immersed into conc. H₂SO₄ for about 30 minutes, and then rinsed with distilled water, and thereafter, is boiled within deionized water for several minutes. Subsequently, aminosilane such as, for example, 1% of distilled trimethoxysilyl propyl diethylenetriamine (DETA) solution or N-(2-amino ethyl)-3-aminopropyl trimethoxy silane (EDA) (in 1 mM acetic acid aqueous solution) and the like is introduced to the substrate 12, and reaction thereof is conducted at a room temperature for about 20 minutes.

This provides immobilizing DETA or EDA onto the surface of the substrate 12. Thereafter, residues are removed by rinsing with distilled water, and it is dried by heating at about 120 degree C. for 3 to 4 minutes within an inert gas atmosphere. Subsequently, the substrate 12 is processed with 1% of m, p-(aminoethyl aminomethyl) phenethyl trimethoxysilane (PEDA) solution (within a mixture of CH₃OH: 1 mM acetic acid aqueous solution at a rate of 95:5) at a room temperature for about 20 minutes, and then rinsed with CH₃OH. Thereafter, it is dried by heating at about 120 degree C. for 3 to 4 minutes within an inert gas atmosphere.

Subsequently, dual functional cross linker such as 1 mM of succinimidyl 4-[maleimide phenyl]butyrate (SMPB) solution and the like is prepared, and then, is dissolved in a small amount of dimethyl sulfoxide (DMSO), and thereafter is diluted with N,N-dimethyl formamide (DMF), DMSO, or a solvent mixture such as a combination of DMSO and C₂H₅OH, or a combination of DMSO and CH₃OH. The substrate 12 is immersed into such diluted solution at a room temperature for about 2 hours, and after rinsed with the diluent solvent, is dried in an inert gas atmosphere.

Having such procedure, ester group in SMPB reacts with amino group in EDA or the like to provide a condition, in which maleimide is exposed on the surfaces of the substrate 12 and the columnar members 14. In such condition, when an oligonucleotide for immobilization 16 having thiol group is introduced in the reaction apparatus 10, thiol group in the oligonucleotide for immobilization 16 reacts with maleimide on the surfaces of the substrate 12 and the columnar members 14, such that the oligonucleotide for immobilization 16 is immobilized on the surfaces of the substrate 12 and the columnar members 14 (see, for example, Chrisey et al., Nucleic Acids Research, 1996, Vol. 24, No. 15, pp. 3031 to 3039). This allows providing the immobilization of the oligonucleotide for immobilization 16 on the surfaces of the substrate 12 and the columnar members 14.

Thereafter, under the condition of elongating the starting template DNA 18, the starting template DNAs 18 are adhered to the surfaces of the substrate 12 and the columnar members 14 (FIG. 2(c)). The starting template DNA 18 may be prepared by a technique, which is similar to that for the starting template DNA for the ordinary PCR, as stated above. When a huge DNA such as chromosomal DNA is employed, for example, first, it is broken by ultrasonic wave to provide DNA fragments, and subsequently the DNA fragment is denatured by alkali or heat to obtain a single strand. When the DNA fragment thus formed into single strand is processed onto the surfaces of the substrate 12 and the columnar members 14, the portion for immobilization in the starting template DNA 18 forms bonds complementary to the oligonucleotide for immobilization 16, as the oligonucleotide for immobilization 16 is immobilized in a the surfaces of the substrate 12 and the columnar members 14. Although a portion of the starting template DNA 18 may be adhere in a shrunk condition or a curled condition as shown in this occasion, if at least a portion thereof is adhered over a plurality of columnar members 14, the subsequent PCR can be move smoothly carried out by employing the starting template DNA 18 as the template.

In order to elongate the starting template DNA 18, the starting template DNA 18 is introduced in the substrate 12 under the condition of, for example, applying a low frequency electric field over the substrate 12. Here, the low frequency electric field may be an electric field of equal to or lower than 100 Hz, for example. This allows to elongate the random-coiled starting template DNA 18. Alternatively, in order to elongate the starting template DNA 18, the starting template DNA 18 is introduced in the substrate 12 under the condition of, for example, applying a high electric field over the substrate 12. Here, the high electric field may be an electric field of equal to or higher than 500 kHz, for example. This produces a dielectrophoresis, thereby providing the elongation of the starting template DNA 18.

Further, in order to elongate the starting template DNA 18, a shear stress may also be utilized. For example, a method of spraying the starting template DNA 18 to adhere thereof on the surfaces of the substrate 12 and the columnar members 14, or a method of creating a flow rate in the reaction field and then introducing the starting template DNA 18 therein to adhere thereof on the surfaces of the substrate 12 and the columnar members 14, or the like may be utilized. A method for creating a flow rate may be that, for example, the starting template DNA 18 is introduced in the reaction field while rotating the substrate 12 to adhere thereof on the surfaces of the substrate 12 and the columnar members 14.

Subsequently, the starting template DNA 18 is immobilized onto the oligonucleotide for immobilization 16 (FIG. 2(d)). For example, psoralen 20 such as 4,5′,8-trimethylpsoralen is employed for immobilizing the starting template DNA 18 on the oligonucleotide for immobilization 16. Psoralen 20 is intercalated between the double strand-sequences of a DNA, and is bound to adjacent pyrimidine base by irradiating light of on about 320 nm to 400 nm, thereby providing a strong binding between the double strands. After such process, residual starting template DNA 18 that is failed to be immobilized on the surfaces of the substrate 12 and the columnar members 14 may be rinsed with, for example, a buffer to be removed away.

In the reaction apparatus 10 which a starting template DNA 18 is immobilized on the surfaces of the substrate 12 and the columnar members 14, a reaction solution prepared by mixing specified quantities of a buffer solution for PCR, primers (sense primer, antisense primer), heat-resistant DNA polymerase and deoxyribonucleotide triphosphate (dNTP: mixture of dATP, dGTP, dCTP and dTTP) is introduced.

After that, the temperature of the reaction field is appropriately controlled to carry out an annealing of the primer over the starting template DNA 18 and an elongation of a complementary strand DNA that complementary to the starting template DNA 18 with a heat-resistant DNA polymerase. After the complementary strand of the starting template DNA 18 is formed, the ordinary PCR process including the denaturation, the annealing and the elongation of the complementary strand is repeated by employing these complementary strands as the template in this time. The complementary strand formed by using the starting template DNA 18 as the template has a length that is capable of providing a function as the template without being immobilized, and then the elongation of the complementary strand can be conducted with higher efficiency. This provides sub-exponentially amplifying the complementary strand to the starting template DNA 18.

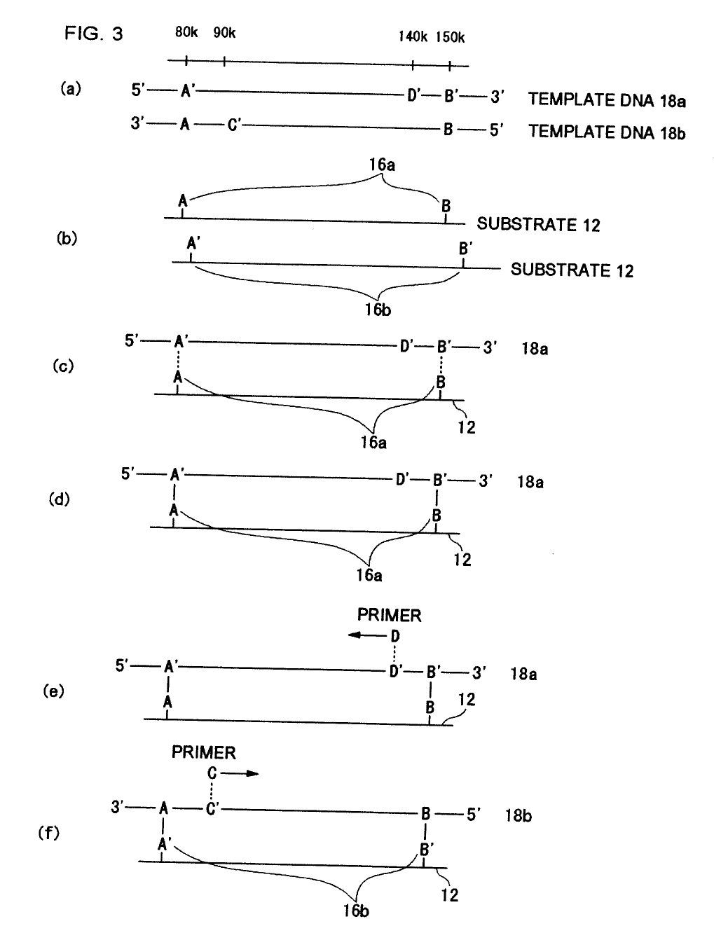

FIG. 3 includes diagrams, schematically illustrating an example of a starting template DNA employed in the present embodiment.

As shown in FIG. 3(a), a starting template DNA is obtained by melting of the double strand comprising of a starting template DNA 18a having DNA sequence A′, D′, B′ from the side of 5′ end and a starting template DNA 18b having DNA sequence B, C′, A from the side of 5′ end. Here, the DNA sequence A′, B′, A and B functions as a portion for immobilization. In addition, the DNA sequence D′ and C′ functions as a starting point of the amplifying target portion and a portion to which the primer is bound. Here, A indicates a sequence complementary to A′, B indicates a sequence complementary to B′, C indicates a sequence complementary to C′ and D indicates a sequence complementary to D′.

FIG. 3(b) is a diagram, showing oligonucleotides for immobilization 16a and 16b that are adhered to the substrate 12. An oligonucleotide for immobilization 16a has DNA sequences A and B complementary to DNA sequences A′ and B′ of the portion for immobilization in the starting template DNA 18a, respectively, for the purpose of adhering the starting template DNA 18a to the substrate 12. An oligonucleotide for immobilization 16b has DNA sequences A′ and B′ complementary to DNA sequences A and B of the portion for immobilization in the starting template DNA 18b, respectively, for the purpose of adhering the starting template DNA 18b to the substrate 12.

FIG. 3(c) shows the condition of the starting template DNA 18a adhered to the oligonucleotide for immobilization 16a. Then, it is reinforced with psoralen 20 to allow the starting template DNA 18a immobilized on the oligonucleotide for immobilization 16a, as shown in FIG. 3(d). Subsequently, as shown in FIG. 3(e), a primer having a DNA sequence D complementary to the DNA sequence D′ of the starting template DNA 18a is introduced to start PCR. Concerning the starting template DNA 18b having a sequence complementary to the starting template DNA 18a, as shown in FIG. 3(f), PCR can be started by introducing a primer immobilized on the oligonucleotide for immobilization 16b and having a sequence C complementary to the DNA sequence C′.

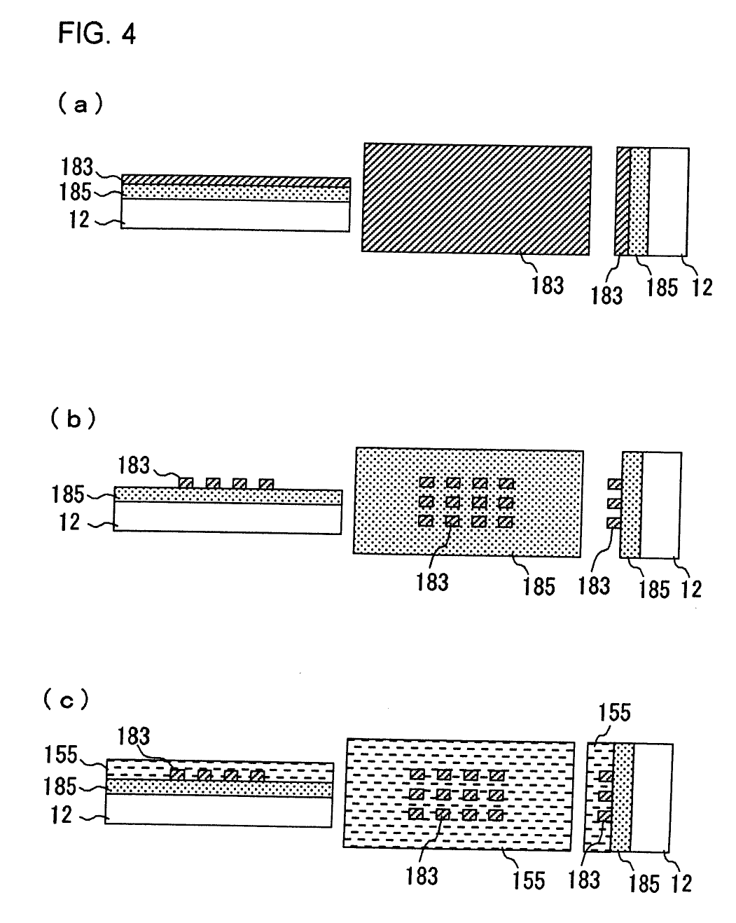

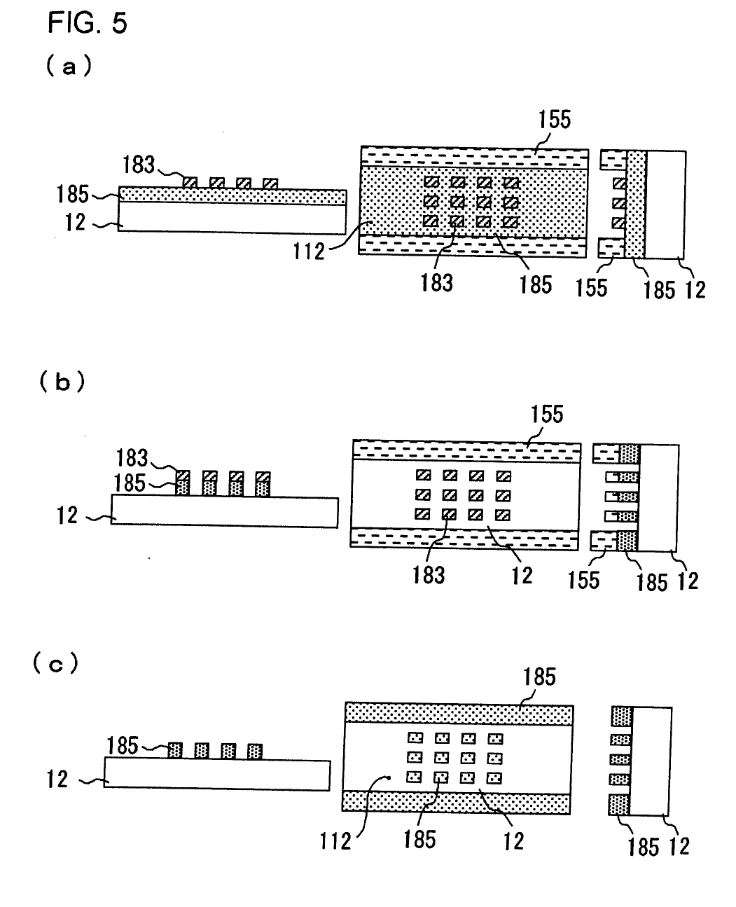

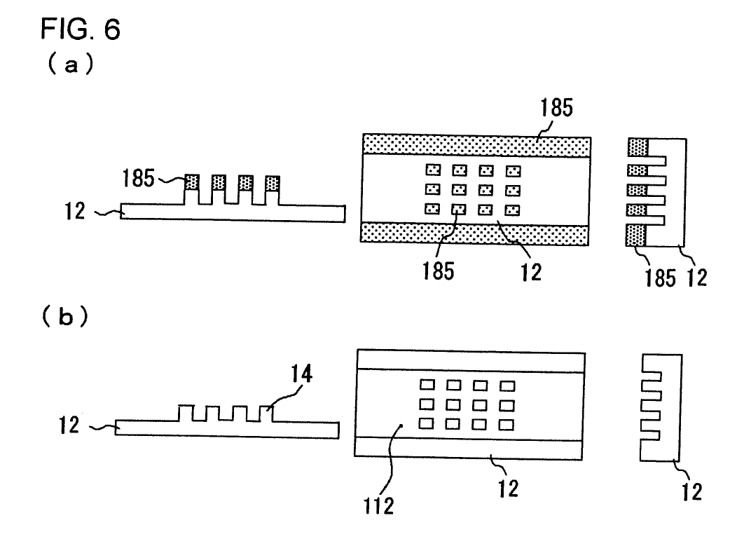

Next, a method for forming the columnar members 14 on the substrate 12 will be described. First, a method for forming the columnar members 14 in the case of composing the substrate 12 of silicon will be described in reference to FIG. 4, FIG. 5 and FIG. 6. While the formation of the columnar members 14 on the substrate 12 may be carried out by etching the substrate 12 into a certain patterned geometry, the method for forming is not particularly limited thereto.

Here, in each of the figures, the center diagram is a plan view, and diagrams of right and left are cross-sectional views. In this method, the columnar members 14 are formed by utilizing a lithographic technology employing a photo resist.

In this case, silicon substrate having a plain orientation (100) is employed for the substrate 12. First, as shown in FIG. 4(a), a silicon oxide film 185 and “sumiregist NEB” (manufactured by Sumitomo Chemical Co., Ltd.) 183 are formed on the substrate 12 in this sequence. The film thicknesses of the a silicon oxide film 185 and the “sumiregist NEB” 183 are 300 nm and 400 nm, respectively. Next, regions to be columnar members 14 are exposed to light. A development process is carried out by using xylene, and a rinse is carried out with isopropyl alcohol. This process provides patterning the “sumiregist NEB” 183, as shown in FIG. 4(b).

Subsequently, a positive photo resist 155 is applied on the entire surface thereof (FIG. 4(c)). Film thickness thereof is set to 1.8 μm. Thereafter, a mask-exposure is conducted so as to expose a region to be a reaction vessel 112 and then carry out the development (FIG. 5(a)).

Then, the silicon oxide film 185 is reactive ion etched (RIE) by using a gaseous mixture of CF₄ and CHF₃.

The thickness of the etched film is set to be 300 nm (FIG. 5(b)). The “sumiregist NEB” 183 is stripped via an organic washing using a mixed solution of acetone, alcohol and water, and then an oxidizing plasma processing is carried out (FIG. 5(c)). Subsequently, the substrate 12 is electron cyclotron resonance (ECR) etched by using HBr gas. A step of the etched silicon substrate (or height of the columnar member) is set to be 3 μm (FIG. 6(a)). Subsequently, a wet etch process is conducted by using buffered hydrofluoric acid (BHF) to remove the silicon oxide film (FIG. 6(b)). The columnar members 14 are formed on the substrate 12 by the above process.

Here, when a plastic material is used for the substrate 12, the formation of the columnar member 14 may be carried out by a known method suitable to the type of the material of the substrate 12 such as etching, compressive molding employing a metal mold such as embossing molding, injection molding, light cure molding and the like.

When the substrate 12 is composed of a plastic material, a master sample is prepared via machining or etching, and a metal mold manufactured by reversely transferred the pattern of the master sample via an electrocasting is employed to conduct an injection molding or an injection compression molding to form the substrate 12 having the columnar members 14 formed thereon. Alternatively, the columnar members 14 may also be formed by a compressive processing by employing a metal mold. Further, the substrate 12 having the columnar members 14 formed thereon may also be formed via a laser beam lithography employing a photopolymeric resin.

### SECOND EMBODIMENT

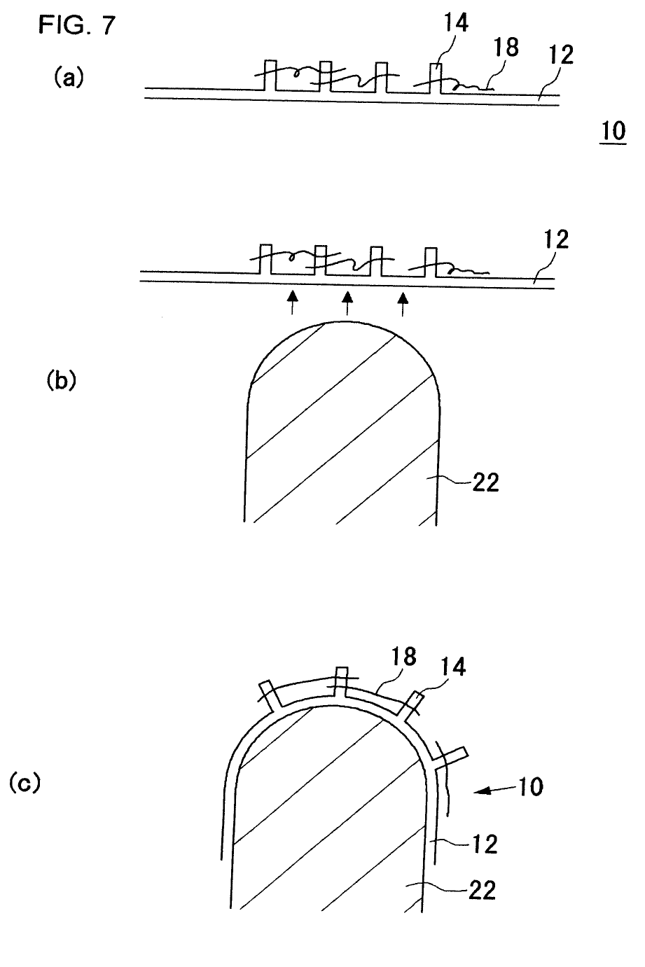

FIG. 7 includes diagrams, illustrating a process for manufacturing a reaction apparatus 10 in second embodiment of the present invention. In the present embodiment, after introducing the starting template DNA 18 on the substrate 12, the substrate 12 is compressively stretched to further elongate the starting template DNA 18 adhered to the columnar member 14. In the present embodiment, the substrate 12 is composed of an elastic material such as various types of stretchable plastic materials, rubbers and the like. Such material includes, for example, poly dimethylsiloxane (PDMS).

As stated above, even though the starting template DNA 18 is adhered and immobilized to the surfaces of the substrate 12 and the columnar members 14 under the elongated condition, a portion of starting template DNA 18 may often be still in a shrunk state as shown in FIG. 7(a). In order to further elongate thereof to conduct PCR with higher efficiency, in the present embodiment, as shown in FIG. 7(b), the substrate 12 is pressurized from the side of the back surface of the substrate 12 by employing a pressurizing member 22 to elongate the substrate 12 as shown in FIG. 7(c). Having this procedure, distances between the columnar members 14 are increased, and the starting template DNA having one end and the other end, both are immobilized to adjacent columnar members 14, respectively, is also be in a state of being elongated.

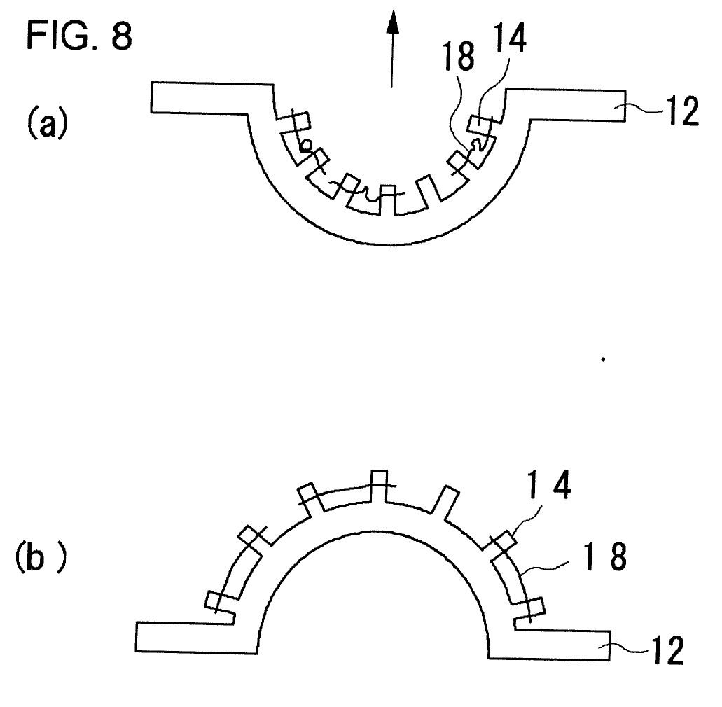

In addition, in the present embodiment, as shown in FIG. 8, the substrate 12 may be provided with concave portions, and the starting template DNA 18 is introduced under the condition of forming the columnar members 14 within the concave portions (FIG. 8(a)), and then, the concave portions of substrate 12 may be inversed after the starting template DNA 18 is introduced (FIG. 8(b)) to achieve an elongation of the surface of the substrate 12 having the columnar members 14 formed thereon.

### THIRD EMBODIMENT

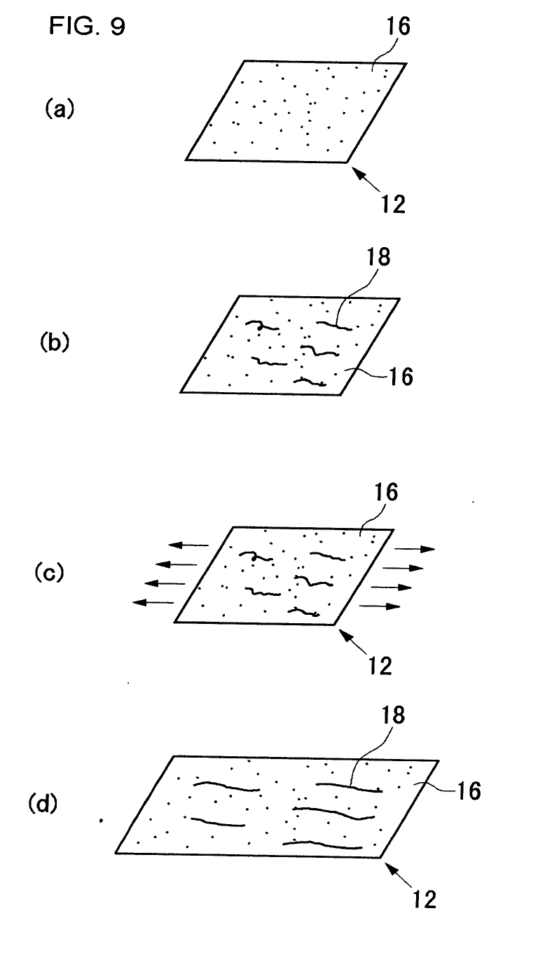

FIG. 9 includes diagrams, illustrating a process for manufacturing a reaction apparatus 10 in the present embodiment of the present invention. The present embodiment is different from first and second embodiments, in terms of providing no columnar member 14 formed on the substrate 12.

First, an oligonucleotide for immobilization 16 is adhered to the surface of the substrate 12 (FIG. 9 (a)). The method for adhering the oligonucleotide for immobilization 16 to the surface of the substrate 12 is similar to that employed in first embodiment. Subsequently, the starting template DNA 18 is immobilized to the substrate 12 (FIG. 9 (b)). Similarly as in first embodiment, the starting template DNA 18 is adhered onto the surface of the substrate 12 under the elongated condition via a method such as, for example, applying a low frequency, applying a high electric field, utilizing a shear stress and the like. Thereafter, similarly as described in first embodiment, the starting template DNA 18 is immobilized to the oligonucleotide for immobilization 16 via a cross linker. While PCR may be conducted in this condition in the present embodiment, similarly as in first embodiment, PCR may also be conducted after stretching the substrate 12 as follows.

As shown in FIG. 9(c), an even force is added to each of both sides of the substrate 12 to stretch the substrate 12. This stretches the substrate 12, and the starting template DNA 18 immobilized onto the substrate 12 surface is also elongated (FIG. 9(d)). When the substrate 12 is stretched in this way, the substrate 12 is preferably composed of a material, which is capable of being uniformly stretched and have no shrinkage-ability after the stretching. As such type of material, PDMS, for example, may be employed.

Having such configuration, PCR can be carried out with a simple method under the condition of elongating the starting template DNA 18.

### FOURTH EMBODIMENT

The present embodiment is different from first to third embodiments, in the point that the oligonucleotide for immobilization 16 is adhered on a surface beads, in stead of the surface of the substrate 12, and after the starting template DNA 18 is immobilized to the oligonucleotide for immobilization 16, and then, the starting template DNA 18 can be elongated by moving beads. In the present embodiment, a Optical Tweezers is employed as a measure for moving beads.

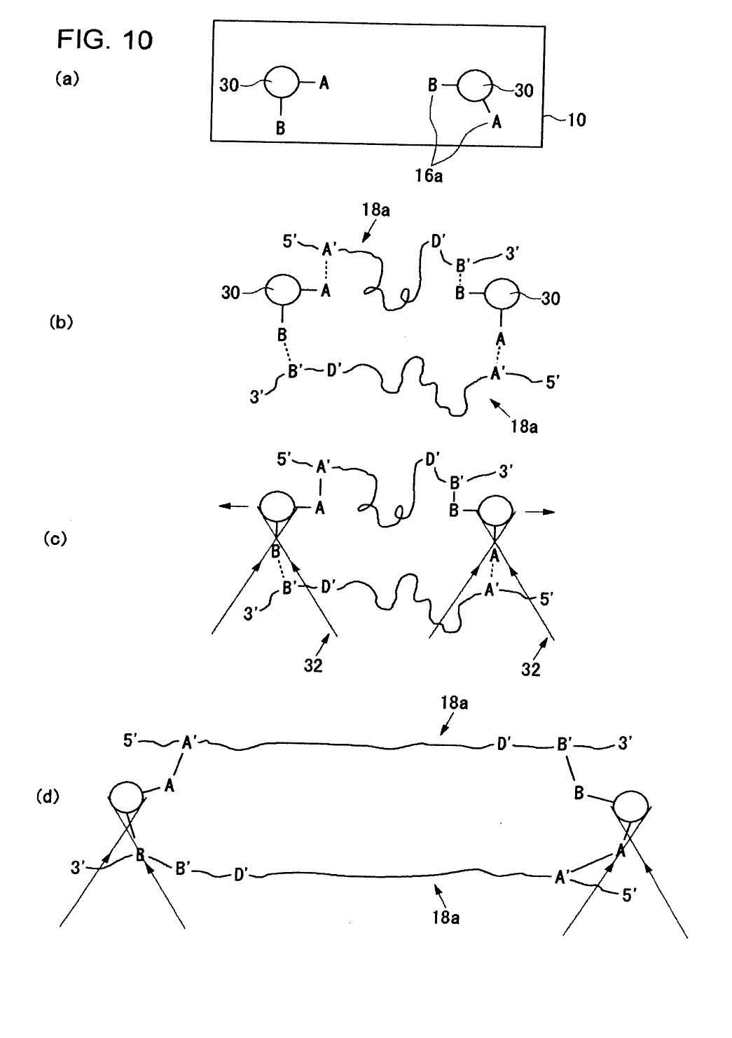

FIG. 10 is a diagram, illustrating a method for immobilizing a starting template DNA 18 in the present embodiment. First, label beads 30 having oligonucleotide for immobilization 16a (DNA sequence A and B) immobilized thereto is introduced into a reaction apparatus 10 (FIG. 10(a)). Available label beads 30 may include, for example, fine particles of polystyrene beads, colloidal gold, latex beads, silica or the like.

Subsequently, the starting template DNA 18a is introduced onto the label beads 30. Having such procedure, the oligonucleotide for immobilization 16a immobilized on the label beads 30 and the portion for immobilization in the starting template DNA 18a (DNA sequence A′ and B′) form a complementary bond (FIG. 10(b)). Thereafter, similarly as described in first embodiment, the oligonucleotide for immobilization 16a is immobilized to the portion for immobilization in the starting template DNA 18a. In this occasion, at least some of the introduced starting template DNAs 18a are bound over two label beads 30 as shown in the diagram.

Subsequently, photo forceps 32 is used to move the label beads 30 (FIG. 10(c)). The photo forceps is a method for capturing a fine material within water in a contact-free and noninvasive manner by a laser beam being condensed by lenses having larger aperture. Accordingly, it is preferable to employ transparent particles having larger diameter than the wavelength of water and having larger refractive index than water for the above-described label beads 30. In addition, metallic fine particles having shorter wavelength than water may also be employed for the label beads 30. This allows catching the label beads 30 with a laser beam. These label beads 30 can be visualized by using an optical microscope, and the label beads 30 can be moved so that the distance between the label beads 30 is presented to be, for example, slightly shorter than the length of the amplifying target portion of the starting template DNA 18. Having this configuration, the starting template DNA 18a bound to over two label beads 30 can be elongated (FIG. 10(d)).

### FIFTH EMBODIMENT

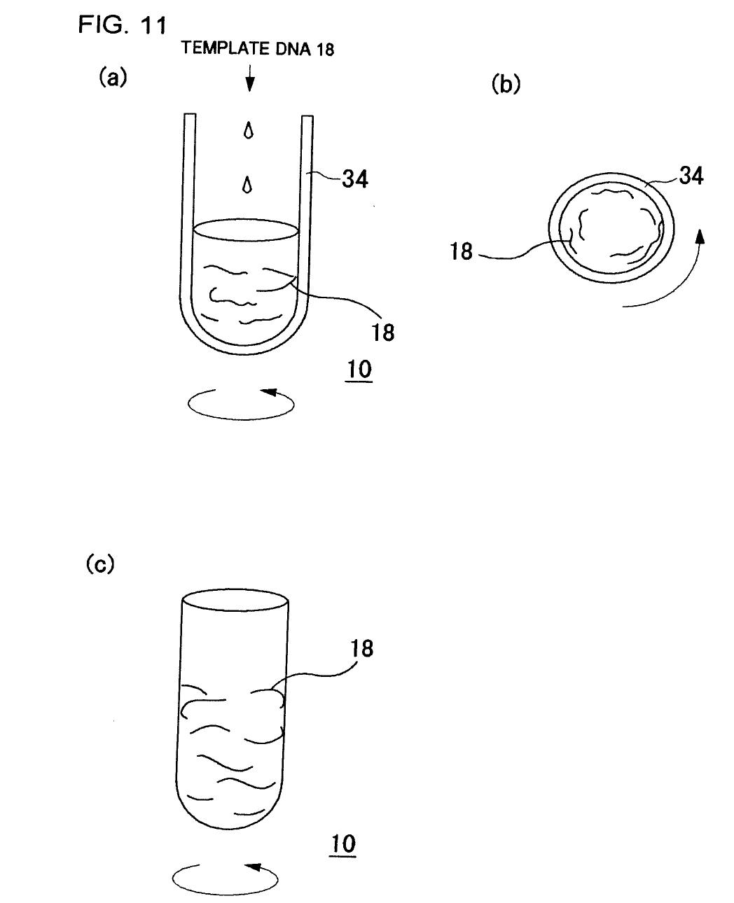

FIG. 11 is a diagram, showing a reaction apparatus 10 in the fifth embodiment of the present invention. Here, a test tube 34 containing therein an oligonucleotide for immobilization (not shown) immobilized thereto can be employed as the reaction apparatus 10. Buffer, for example, is introduced in the test tube 34 and the test tube 34 is spun, and while maintaining the condition thereof, a solution prepared by dissolving the starting template DNA 18 in the buffer is introduced in the test tube 34 (FIG. 11(a)). Here, since the test tube 34 is spun, a shear stress is exerted over the introduced starting template DNA 18, and thus the starting template DNA 18 is adhered onto the sidewall of the test tube 34 under the elongated condition (FIG. 11B). Thereafter, the buffer is removed via a vacuum removal process or the like to obtain the reaction apparatus 10 having the starting template DNA 18 immobilized onto the side wall of the test tube 34 (FIG. 11 (c)).

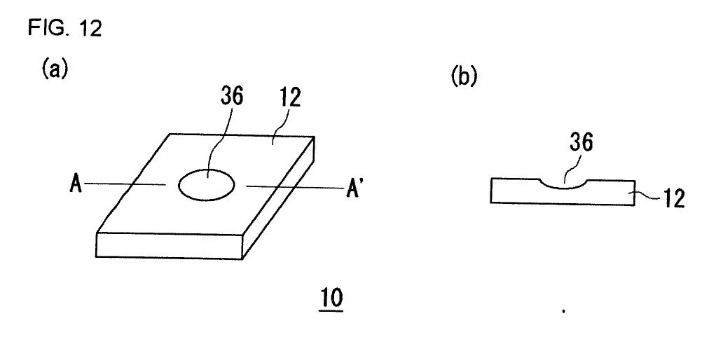

FIG. 12 is a diagram, showing another example of the reaction apparatus 10 in the present embodiment. Here, the starting template DNA 18 can be introduced in a well 36 formed on the substrate 12 (FIG. 12 (a)). FIG. 12 (b) is a cross-sectional view of FIG. 12 (a) along line A-A′. In this case, the starting template DNA solution is introduced in the well 36 while spinning the substrate 12. Having this configuration, the reaction apparatus 10 having the starting template DNA 18 immobilized onto the sidewall of the well 36 can be obtained.

### SIXTH EMBODIMENT

While configurations, in which the starting template DNA 18 contains the amplifying target portion and the portion for immobilization located in the outside of the amplifying target portion, and the complementary strand is synthesized by using the amplifying target portion as the template under the condition of the portion for immobilization being immobilized in the above-described first to fifth embodiments, the complementary strand can also be synthesized by employing the portion for immobilization in the starting template DNA 18 as the amplifying target. Having such configuration, the complementary strand synthesized by using the starting template DNA 18 as the template also has a portion for immobilization, and thus the synthesized complementary strand can be immobilized to the surface of the substrate 12, thereby providing efficient amplification of longer DNA strand.

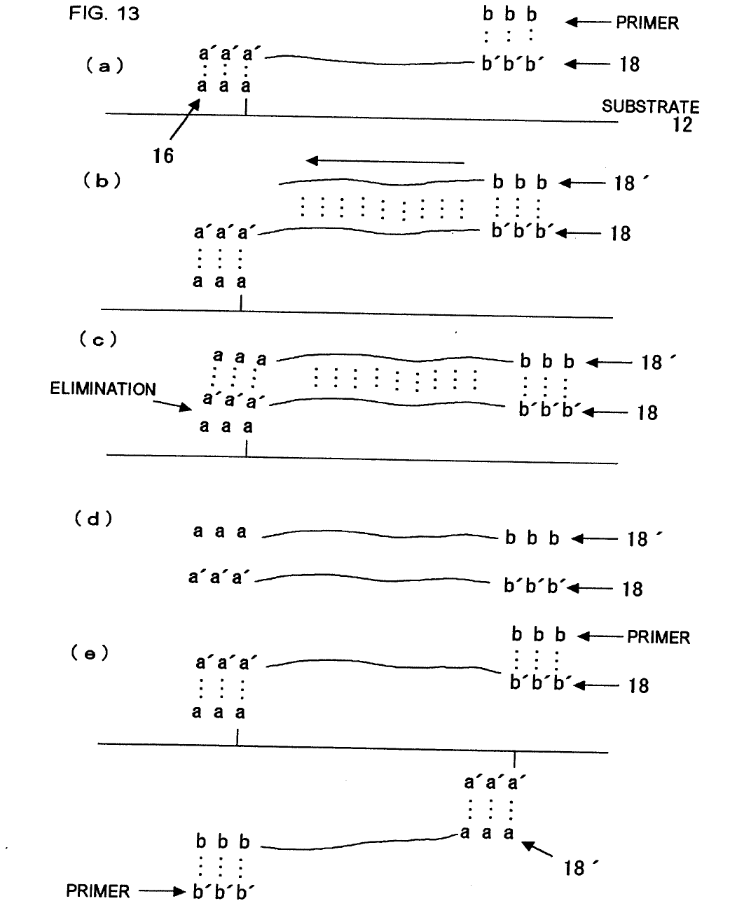

FIG. 13 includes diagrams, illustrating a method for amplifying a starting template DNA 18 in the present embodiment. As shown in FIG. 13(a), the starting template DNA 18 includes a sequence a′a′a′ and a sequence b′b′b′. On the substrate 12, an oligonucleotide for immobilization 16 having a sequence aaa complementary to the sequence a′a′a′ in the starting template DNA 18 is immobilized. Here, the sequence a′a′a′ and the sequence b′b′b′ are located at respective ends of the amplifying target portion of the starting template DNA 18. Under such condition, a primer containing a sequence bbb complementary to the sequence b′b′b′ is introduced to start PCR.

As shown in FIG. 13B, the primer having the sequence bbb creates hydrogen bond with the sequence b′b′b′ in the starting template DNA 18, and a complementary strand complementary to the starting template DNA 18 is synthesized via PCR. The synthesis of the complementary strand via PCR is conducted at about 70 degree C., for example. Subsequently, the temperature in the reaction vessel is increased to about 75 to 85 degree C. in a stage of proceeding the PCR process to a certain extent, so that bonds of the oligonucleotide for immobilization 16 having the sequence aaa with the sequence a′a′a′ in the starting template DNA 18 are deactivated and a strand complementary to the sequence a′a′a′ in the starting template DNA 18 is also synthesized, thereby obtaining a secondary template DNA 18′, which is a complementary strand to the starting template DNA 18 (FIG. 13(c)).

While the starting template DNA 18 and the secondary template DNA 18′ are longer DNA strands of equal to or higher than several kb, the oligonucleotide for immobilization 16 is a very short DNA strand of on the order of several tens to several hundreds b, as compared with the length of the starting template DNA 18. Since shorter DNA strand has weaker binding strength between double strands than longer DNA strand, bonds between the double strands are easy to be deactivated by an influence of molecular energy created when heated. Therefore, in the present embodiment, before a denaturation process for separating the synthesized secondary template DNA 18′ from the starting template DNA 18 in PCR process, a heat of a temperature lower than the temperature set in the denaturation process is added to separate the starting template DNA 18 from the oligonucleotide for immobilization 16 while elongating the secondary template DNA 18′, under the condition of binding the starting template DNA 18 with the secondary template DNA 18′, such that the secondary template DNA 18′ can be synthesized.

Subsequently, the temperature in the reaction vessel is increased to about 90 to 98 degree C. to denature the starting template DNA 18 and the secondary template DNA 18′, thereby forming a single strand (FIG. 13(d)). Here, as described in first embodiment, elongation of the starting template DNA 18 and the secondary template DNA 18′ is conducted by using an electric field-applying unit that applies low frequency electric field and high electric field and a shearing stress-applying unit that applies shearing stress to the reaction field.

Since secondary template DNA 18′ is synthesized so as to contain the sequence aaa complementary to the sequence a′a′a′ in the starting template DNA 18, the synthesized secondary template DNA 18′ can be immobilized to the substrate 12 under the elongated condition, by immobilizing on substrate 12 the oligonucleotide for immobilization 16 having the sequence a′a′a′, as shown in FIG. 13(e). Subsequently, a primer having a sequence bbb and a primer having a sequence b′b′b′ are introduced to synthesize respective complementary strands by employing the starting template DNA 18 and the secondary template DNA 18′ as the templates, respectively.

The above-mentioned process is repeated to allow sequentially amplifying the starting template DNA 18. Since the synthesized DNA strand is immobilized onto the substrate 12 in the present embodiment, relatively longer DNA strand can be exponentially synthesized with higher efficiency.

### SEVENTH EMBODIMENT

The method for elongating the template DNA and the method for immobilizing thereof onto the substrate described in the above embodiments can be also applied to a technology for breaking a DNA strand at a specified site. A method for immobilizing a DNA strand onto a substrate so that the intended site of the DNA strand for breaking contacts with deoxyribonuclease (DNase) to enhance a probability that the site is specifically broken will be described in the present embodiment.

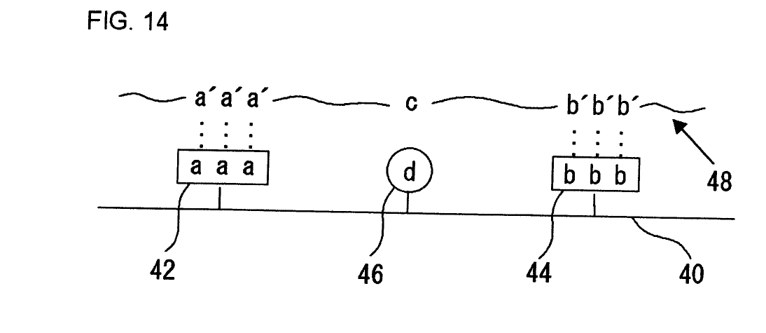

FIG. 14 is a conceptual diagram, illustrating a method for immobilizing a DNA strand in the present embodiment. Here, a breaking target DNA 48 to be broken includes a sequence a′a′a′ and a sequence b′b′b′, which are portions for immobilization are included, as shown. An oligonucleotide for immobilization 42 containing a sequence aaa and an oligonucleotide for immobilization 44 containing a sequence bbb are immobilized to a substrate 40. The sequence aaa is complementary to the sequence a′a′a′, and the sequence bbb is complementary to the sequence b′b′b′. On the substrate 40, between the oligonucleotide for immobilization 42 and the oligonucleotide for immobilization 44, a nicking enzyme 46 is immobilized at a position corresponding to a site c to be broken of the DNA 48 to be broken when the breaking target DNA 48 to be broken is immobilized to the oligonucleotide for immobilization 42 and to the oligonucleotide for immobilization 44 under the elongated condition. By composing the substrate 40 in this way, the probability that the site c to be broken contacts with the nicking enzyme 46 is increased when the DNA 48 to be broken is immobilized onto the substrate 40, and thus breaking thereof can be effectively conducted at the specified site. The nicking enzyme 46 is DNase.

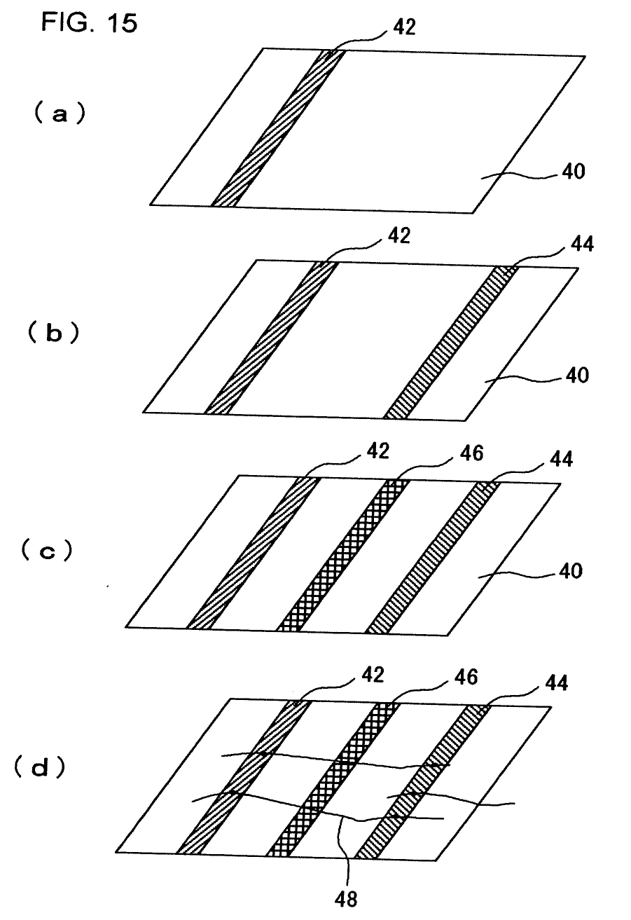

FIG. 15 includes process diagrams, illustrating a method for manufacturing a substrate 12 in the present embodiment.

First, as shown in FIG. 15(a), an oligonucleotide for immobilization 42 is adhered onto a substrate 40 into a strip. Subsequently, as shown in FIG. 15(b), an oligonucleotide for immobilization 44 is adhered thereto into a strip, spaced apart from the oligonucleotide for immobilization 42 with a certain distance. The space between the oligonucleotide for immobilization 42 and the oligonucleotide for immobilization 44 may be preferably equivalent to or slightly shorter than the distance between the portions for immobilization in the DNAs 48 to be broken. Thereafter, as shown in FIG. 15(c), the nicking enzyme 46 is adhered into a strip to a position where the site to be broken is located when the DNA 48 to be broken is elongated and immobilized to the oligonucleotide for immobilization 42 and the oligonucleotide for immobilization 44, Thus formed DNA 48 to be broken is introduced in the substrate 40 to bind the portion for immobilization in the DNA 48 to be broken with the oligonucleotide for immobilization 42 and the oligonucleotide for immobilization 44, thereby immobilizing the DNA 48 to be broken to the substrate 40.

Since the site to be broken of the DNA 48 to be broken is disposed in the vicinity of the nicking enzyme 46 on the substrate 40 in this occasion, the site to be broken of the DNA 48 to be broken is effectively broken with the nicking enzyme 46.

While immobilizations of the oligonucleotide for immobilization 42, the oligonucleotide for immobilization 44 and the nicking enzyme 46 to the substrate 40 may be carried out in various ways, some exemplary methods will be illustrated. As an example, when the substrate 40 is composed of silicon, the oligonucleotide for immobilization 42, the oligonucleotide for immobilization 44 and the nicking enzyme 46 can be immobilized to the substrate 40 via silane coupling agent that is similar to ones described in first embodiment such as aminosilane and the like. First, silane coupling agent is introduced selectively to locations on the surface of the substrate 40 where oligonucleotide for immobilization 42 is to be immobilized, and the oligonucleotide for immobilization 42 is immobilized to the substrate 40 via silane coupling agent. Subsequently, similar silane coupling agent is introduced selectively to locations on the surface of the substrate 40 where oligonucleotide for immobilization 44 is to be immobilized, and the oligonucleotide for immobilization 44 is immobilized to the substrate 40 via silane coupling agent. Then, similar silane coupling agent is introduced selectively to locations on the surface of the substrate 40 where the nicking enzyme 46 is to be immobilized, and the nicking enzyme 46 is immobilized to the substrate 40 via silane coupling agent.

Alternatively, as another example, hydrophobic regions are formed on the surface of the substrate 40, and then the oligonucleotide for immobilization 42, the oligonucleotide for immobilization 44 and the nicking enzyme 46 are sequentially adhered on other regions except the hydrophobic regions. In this case, the substrate 40 is composed of, for example, glass, or the surface of the substrate 40 is coated with a silicon oxide film, or coated with a metal. In this state, clean condition is provided to the surface of the substrate 40, and then hydrophobicity is provided to regions other than the regions of the substrate 40, to which the oligonucleotide for immobilization 42 is immobilized. The process for providing hydrophobicity to the surface of the substrate 40 may be conducted by employing a printing technology such as stamping or ink-jet printing. In the method by using stamping, polydimethylsiloxane (PDMS) resin is employed. PDMS resin is conducted by polymerizing silicone oil, and a condition of containing silicone oil filled within spaces in molecule is maintained after the resinification. Therefore, when PDMS resin is in contact with a hydrophilic surface such as, for example, a glass surface, the contacted portion acquires stronger hydrophobicity and thus repels water. By utilizing such characteristic, PDMS block having a concave portion corresponding to the region where the oligonucleotide for immobilization 42 is to be formed is employed as a stamp and is in contact with the hydrophilic substrate 40 to provide hydrophobicity to regions other than the regions where the oligonucleotide for immobilization 42 is to be formed.

In the method by using ink-jet printing, a type of silicone oil having lower viscousness is employed as an ink for the ink-jet printing, a pattern is printed so that silicone oil is adhered to regions in the surface of the substrate 40 other than the regions where the oligonucleotide for immobilization 42 is to be formed.

After the oligonucleotide for immobilization 42 is adhered on the surface of the substrate 40, silicone oil or the like applied on the surface of the substrate 40 is washed away using an organic solvent, and then the oligonucleotide for immobilization 44 is adhered onto the surface of the substrate 40 via the method same as that employed for adhering the oligonucleotide for immobilization 42 to the surface of the substrate 40. Thereafter, silicone oil or the like applied on the surface of the substrate 40 is washed away again by using an organic solvent.

Subsequently, the nicking enzyme 46 is adhered onto the relevant locations on the surface of the substrate 40 by employing a solution including the nicking enzyme 46 as the ink for the ink-jet printing process.

Here, the oligonucleotide for immobilization 42 and the oligonucleotide for immobilization 44 may equally be adhered onto the surface of the substrate 40 via the ink-jet printing process, by employing a solution including the oligonucleotide for immobilization 42 and a solution including the oligonucleotide for immobilization 44 as ink, respectively. The ink for the ink-jet printing process may preferably include an antiseptic for preventing denaturation of the nicking enzyme 46, the oligonucleotide for immobilization 42, oligonucleotide for immobilization 44 or the like.

In addition, in the present embodiment, protruding portions such as columnar members may be formed to provide a configuration, in which DNA 48 to be broken is immobilized to the protruding portions, similarly as described in first embodiment.

In addition, in the first embodiment, the technology of the ink-jet printing process may equally be employed similarly as described in the seventh embodiment, to provide a pattern of the oligonucleotide for immobilization disposed with a certain distances, instead of forming columnar member 14. In addition, in the above-described first to seventh embodiments, the immobilization of the oligonucleotide for immobilization to the substrate may be carried out by using various types of known technologies such as photo lithography and the like, as well as the methods stated above

### EXAMPLES

The present invention will be described in reference to examples as follows, though the present invention is not limited thereto.

In the present example, genomic DNA of a nematode (C. elegans) was employed for a starting template DNA 18. The nematode has 16,000 to 19,000 genes, and in the present example, a region of 50 kb (around 90 k to 140 k in FIG. 3(a)) including from genes tpa-1 to daf-1 in fourth chromosome of these genes is amplified. In this case, two locations of around 80 k and around 150 k were selected for the portions for immobilization in the starting template DNA 18.

In the present example, following sequences A and B were employed as the oligonucleotides for immobilization 16. In addition, following sequences C and D were employed as the primers (sense primer, antisense primer).

A: 5′-SH-agcttacgacaaaatgcacaaattcacaaaattt-3′ (SEQ ID NO: 1);

B: 5′-SH-gcgtcattattctgatggttatctttttgagaggt-3′ (SEQ ID NO: 2);

C: 5′-actttcccacacttgataaatatcctcg-3′ (SEQ ID NO: 3); and

D: 5′-ataatcgttttcaaccgcaaaattacag-3′ (SEQ ID NO: 4).

### Example 1

An reaction apparatus 10 having columnar members 14 formed on a surface of a substrate 12 was manufactured via a method described in first embodiment. In this case, the substrate 12 was composed of a silicon substrate having (100) plane as a principal plane. The columnar members 14 were formed via a method described in FIG. 4 to FIG. 6. In this case, the columnar members 14 were formed to provide distances d therebetween of about 20 μm. Then, EDA was immobilized onto the surfaces of the substrate 12 and the columnar members 14, and then, SMPB was immobilized to EDA via the method described in first embodiment. Thereafter, an oligonucleotide for immobilization in the above-described sequences A and B were introduced to immobilize the oligonucleotides for immobilization in the sequence A and the sequence B to the substrate 12 and the columnar member 14.

A starting template DNA 18 was introduced on the substrate 12 and the columnar member 14 under a condition of applying a low frequency electric field of about 10 Hz over the substrate 12.

Subsequently, TEN buffer (10 mM tris (pH 7.6), 1 mM ethylene diamine tetraacetic acid (EDTA) solution and 50 mM NaCl) was introduced into the reaction apparatus 10, and then ethanol solution of 4,5′,8-trimethylpsoralen was introduced into the reaction apparatus 10, and after intercalating thereof for about 2 minutes, light of about 365 nm was irradiated for about 20 minutes by using an ultra violet (UV) apparatus (manufactured by UVP). Thereafter, the surface of the substrate 12 was cleaned with the buffer solution to remove the starting template DNA, which had not been immobilized onto the surface of the substrate 12 or the surfaces of the columnar members 14.

Subsequently, PCR reaction is conducted via TaKaRa LA PCR™ method. As described above, an appropriate number of the reaction apparatus 10 of about 3 mm square having the starting template DNA immobilized thereto was transferred to a fine centrifugation vessel.

10 μl of 10×LA PCR buffer II (free of Mg2+), 10 μl of 25 mM-MgCl₂, 16 μl of dNTP mixture (each components: 2.5 mM), respective 1 μl (100 pmol/μl) of primers (sense primer, antisense primer) and 1 μl of TaKaRa LA Taq were added therein, and then sterilized distilled water was added thereto to obtain a total amount of 100 μl. Thereafter, a denaturation reaction was conducted at 94 degree C. for one minute by employing a thermal cycler, and then, 14 cycles of a reaction cycle composed of a denaturation at 98 degree C. and for 20 seconds and an annealing of the primer and an elongation of a complementary strand DNA at 68 degree C. and for 20 minutes; and 16 cycles of a reaction cycle composed of a denaturation at 98 degree C. and for 20 seconds and an annealing of the primer and an elongation of a complementary strand DNA at 68 degree C. and for 20 minutes and 15 seconds; were carried out, and eventually the mixture was reacted at 72 degree C. for 10 minutes.

An electrophoresis of the product obtained by the above-described reaction was conducted in a 0.4%, high strength type of agarose gel, and an amplified band around 50 kbp was observed.

### Example 2

The reaction apparatus 10 was manufactured via the method described in third embodiment. This case is different from example 1, in terms of having no columnar member 14 formed on the surface of the substrate 12. The substrate 12 was composed of a silicon substrate having (100) plane as a principal plane, similarly as in example 1. Similarly as in example 1, PCR was conducted while a starting template DNA was immobilized onto the surface of the substrate 12. As a result, an electrophoresis of the product obtained by the above-described reaction was also conducted in a 0.4%, high strength type of agarose gel in the present example, and an amplified band around 50 kbp was also observed.

1. A method for amplifying a DNA fragment, including:
binding said DNA fragment to a binding site formed on a surface of a base member; and
synthesizing a complementary strand of said DNA fragment by using said DNA fragment as a template under a status of said DNA fragment being bound to said surface of the base member.

2. The method for amplifying the DNA fragment according to claim 1, wherein said binding site is configured to be bound to a DNA sequence, which is located in the outside of an amplifying target region of an amplifying target DNA fragment.

3. The method for amplifying the DNA fragment according to claim 1, wherein said binding site contains two or more types of oligonucleotides for immobilization, said oligonucleotides having sequences complementary to DNA sequences located in the both outsides of an amplifying target region of an amplifying target DNA fragment.

4. The method for amplifying the DNA fragment according to claim 1, wherein said synthesizing the complementary strand includes denaturing and separating said template DNA fragment and said complementary strand, and wherein in said binding said DNA fragment, said DNA fragment is immobilized to said binding site so that said DNA fragment is not separated from said binding site in said denaturing and separating.

5. The method for amplifying the DNA fragment according to claim 1, wherein
said binding site is configured to be bound to a portion of the amplifying target region of the amplifying target DNA fragment, and said method further including,
in said synthesizing the complementary strand, deactivating a bond of said DNA fragment with said binding site.

6. The method for amplifying the DNA fragment according to claim 1, wherein said binding site includes an oligonucleotide for immobilization having a sequence complementary to a portion of the amplifying target DNA fragment.

7. The method for amplifying the DNA fragment according to claim 1, wherein said DNA fragment is bound to said the surface of the base member under an elongated condition in said binding said DNA fragment.

8. The method for amplifying the DNA fragment according to claim 7, wherein said DNA fragment is bound to the surface of said base member under an elongated condition by applying a low frequency electric field over said DNA fragment in said binding said DNA fragment.

9. The method for amplifying the DNA fragment according to claim 7, wherein said DNA fragment is bound to the surface of said base member under an elongated condition by applying a high electric field over said DNA fragment in said binding said DNA fragment.

10. The method for amplifying the DNA fragment according to claim 1, further comprising stretching the surface of said base member after said binding said DNA fragment.

11. The method for amplifying the DNA fragment according to claim 1, wherein said amplifying target DNA fragment has a length of equal to or larger than 10 kb.

12. A reaction apparatus for conducting an amplifying of a DNA fragment, comprising:
a surface of a base member; and
binding sites that are formed on said surface of said base member and are capable of being bound to amplifying target DNA fragments.

13. The reaction apparatus according to claim 12, wherein
said binding sites are formed on a plurality of regions provided with certain distances therebetween.

14. The reaction apparatus according to claim 12, wherein said binding site includes a plurality of protruding portions formed on said surface of the base member.

15. The reaction apparatus according to claim 12, wherein said binding site includes an oligonucleotide for immobilization having a sequence complementary to a portion of the amplifying target DNA fragment.

16. The reaction apparatus according to claim 12, said binding site is configured to be bound to DNA sequences located in the both sides of an amplifying target region of an amplifying target DNA fragment.

17. The reaction apparatus according to claim 12, said base member is composed of a material, which is capable of being stretched.

18. A method for manufacturing a reaction apparatus for conducting an amplification of a DNA fragment, comprising:
forming a binding site, which is to be bound to an amplifying target DNA fragment, on a surface of a base member; and
binding said amplifying target DNA fragment to said binding site.

19. The method for manufacturing the reaction apparatus according to claim 18, wherein an oligonucleotide for immobilization having a sequence complementary to a portion of the amplifying target DNA fragment is immobilized on the surface of said base member in said forming said binding site.

20. The method for manufacturing the reaction apparatus according to claim 19, wherein said DNA fragment is bound to the surface of said base member under an elongated condition in said binding said DNA fragment.

21. The method for manufacturing the reaction apparatus according to claim 19, further comprising stretching the surface of said base member after said binding said DNA fragment.
